(19) United States
(12) Patent Application Publication
(10) Pub. No.: US 2020/0086097 A1
(43) Pub. Date: Mar. 19, 2020

(54) PUMP SPRAY OR PRESSURIZED AEROSOL DEVICE WITH APPLICATOR ARM

(71) Applicant: NANOMETICS LLC, New York, NY (US)

(72) Inventors: Brooke Bullock Lao, Leesville, SC (US); Andrew Mahon, New York, NY (US)

(21) Appl. No.: 16/468,606

(22) PCT Filed: Dec. 13, 2017
(86) PCT No.: PCT/US17/66143

§ 371 Date: Jun. 11, 2019

Related U.S. Application Data
(60) Provisional application No. 62/443,643, filed on Jan. 6, 2017.
(60) Provisional application No. 62/436,115, filed on Dec. 19, 2016.

(51) Int. Cl. A61M 35/00; A61N 5/06

(52) U.S. Cl. CPC A61M 35/003; A61N 5/062

(57) ABSTRACT

A spray device for dispensing a product includes a canister, a wand attached to the canister by a ball joint, a manually actuatable valve connecting the canister to the wand for dispensing a product through a nozzle at a free end of the valve. The wand may be telescoping. Applicator attachments are provided for securing to the free end of the wand for varying the delivery.

### CROSS-REFERENCE TO RELATED APPLICATIONS

This application claims priority to U.S. Provisional Application No. 62/433,643 filed 13 Dec. 2016 and U.S. Provisional Application No. 62/436,115 filed 19 Dec. 2016, each of which applications is incorporated by reference in its entirety herein.

### BACKGROUND OF THE INVENTION

The present invention is generally an apparatus for delivering a sprayable aerosol or non-aerosol liquid, solid, colloidal suspension, or gas and consists of a supply canister, extension applicator attached to the canister, a trigger that releases product from the canister, a cap attached to the supply canister, a tube that carries the product to the spray nozzle at the terminus of the applicator, a valve, and a toggle switch that locks the trigger.

### BACKGROUND OF THE INVENTION

The present Invention generally relates to apparatus and methods for the self-application of sprayable liquid, solid, colloidal suspension, or gas to the human body, such as skin care products, or topical medications, drugs and treatments, especially to less accessible parts of the body, such as the back and shoulders. In addition, the invention is useful for people with physical handicaps, the elderly, those who live alone, and others with limited mobility who may have trouble applying topical formulas to the lower extremities and limbs, such as feet and legs.

The ergonomics of most sprayable devices and products do not facilitate the self-application of the spray formula to less accessible parts of the body, such as the back, shoulders, and lower extremities. The development of a device that facilitates the self-application of topical products and medications to less accessible areas of the body remains a priority and is addressed with the present invention.

### SUMMARY OF THE INVENTION

The following detailed description is of the best currently contemplated modes of carrying out the invention. This description is not meant to be in a limited sense, but to outline the inventions general principles and uses of the present invention. Presented here are examples of ways the invention may be constructed, and the inventions breadth is best described by the appended claims.

Broadly, the present invention provides apparatus methods for the delivery and self-application of topical formulas to the human body, and examples of cases for which the invention would be well-suited. In particular, the present invention may be used to facilitate self-application of the liquids, as the size and lightweight construction of the delivery device of the present invention enables a user to easily reach all locations on the body, including harder-to-reach areas such as the back, shoulders, and lower extremities.

Most sprayable devices dispense topical formulas through a spray nozzle that is located immediately atop a supply canister, however these spray products limit the user's ability to self-apply formula to all areas of the body. The present invention is unique because it separates the spray nozzle from the supply canister by an extension arm, and significantly improves the user's ability to self-apply product to otherwise inaccessible areas.

An apparatus for delivering liquid, fluid, solid (powder), colloidal suspension, or gas comprises, in accordance with the present invention, (a) a supply canister adapted to contain the liquid, fluid, solid, colloidal suspension, or gas, (b) a cap attached to the top of the supply canister, (c) a hinging or rotatable ball joint mechanism, (d) for non-aerosol pump versions, a dip tube extending to the bottom of the canister attached to a 360° valve with a ball that allows the valve to spray when inverted that is attached to the manual pump spray actuator, (e) an extension applicator arm having an attached applicator end and a terminus applicator end, the extension arm is attached to the canister cap by the hinging or rotatable ball joint mechanism that allows the extension arm to be selectively pivoted longitudinally and/or laterally (in elevation and/or in azimuth) with respect to the canister cap. The extension arm define a chamber which may be of fixed or adjustable length extending longitudinally through the extension arm. The apparatus further comprises (f) an actuator adapted to release the liquid fluid, solid, colloidal suspension, or gas contained in the supply canister or a manual pump spray actuator adapted to mechanically move the contents of the supply canister, and (g) a tube extending through the chamber and the hinging or rotatable ball joint mechanism and having a attached tube end connected to the actuator and a terminus tube end disposed at the terminus extension arm end to carry the canister's contents to the terminus extension applicator end. The tube may be a liner or interior hollowed section that can be collapsed with the arm, in the case that the arm is telescoping.

In accordance with the present invention, a device for holding and dispensing housing fluid, solid, colloidal suspension, or solid includes a canister housing that may be a traditional aerosol container, a bag-on-valve aerosol container (e.g., bag or pouch attached to manual or mechanical pump actuator), or a pump spray container with a pump mounted on the neck of a rigid canister or with a dip tube. A canister cap is attached to the supply canister. The cap may have dispensing button, a trigger, or a manual pump actuator. The cap serves in part to connect the canister to an extension applicator arm. The device includes a hinge for the extension applicator arm. The hinge may accommodate a vertical or horizontal motion (in elevation and azimuth) by utilization of a 360° valve. The hinge may be replaced with a rotatable ball joint mechanism. For a non-aerosol pump version, the device incorporates a 360° valve housing a ball that allows the valve to spray at any angle, even when inverted. The valve is connected to a dip tube that extends to the bottom of the canister and carries the canisters contents to the valve. An extension applicator arm is attached to the cap via the living hinge. The extension applicator arm may be rigid and of a fixed length, telescoping, or flexible. The extension applicator arm ranges in length depending on the size of the housing canister, typically from 3 to 10 inches.

The canister typically has a lateral cylindrical surface and the extension arm is movable between a first and a second position by means of the hinge or rotatable ball joint member, the arm being disposed adjacent to the lateral cylindrical surface in the first position in parallel with the lateral cylindrical surface.

### BRIEF DESCRIPTION OF THE DRAWINGS

FIG. 1 is a schematic side elevational view, partly in cross-section, of a dispenser for fluidic material, in accordance with the present invention.

FIG. 2 is a schematic side elevational view, partly in cross-section, of another dispenser for fluidic material, in accordance with the present invention.

FIGS. 3A-3C are each a schematic side elevational view of a respective applicator arm that may be incorporated as a components of a dispenser in accordance with the invention.

FIGS. 4A-4C are schematic longitudinal cross-sectional views of the applicator arms shown in FIGS. 3A-3C, respectively.

FIGS. 5A-5C are schematic perspective or isometric views of applicator pads that are connectable to a distal or free end of an applicator arm, in accordance with the present invention.

FIG. 6 is a schematic partial side elevational view of a dispenser actuator assembly, in accordance with the present invention, that may be incorporated into a fluid dispenser device as described herein.

FIG. 7 is a schematic side elevational view of a further dispenser for fluidic material, in accordance with the present invention.

FIG. 8 is a schematic side elevational view of yet another dispenser for fluidic material, in accordance with the present invention.

FIG. 9A-9D are schematic side elevational views of alternative distal or free ends of flexible applicator arms included in the dispensers of FIGS. 7 and 8.

### DETAILED DESCRIPTION

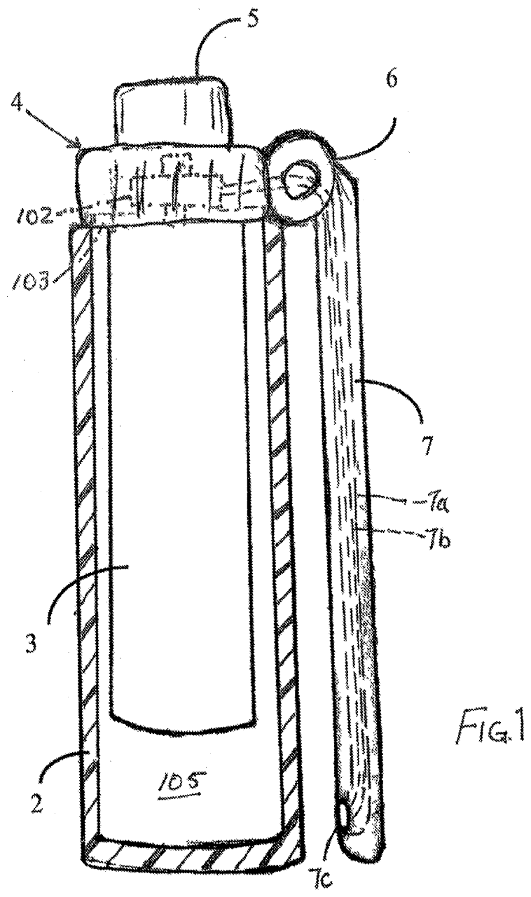

FIG. 1 depicts a dispenser 1 with a pump or actuator 102 mounted to a neck 103 of a rigid container or canister 2. The pinup or actuator 102, which may be spring-loaded, is sealed and fixed to the neck within 103 a cap 4. The pump or actuator 102 is operated by pressing a button 5, whereupon air enters through an inlet (not shown) into a space 105 separating a pouch or bag 3 from an inner wall of the canister 2 so that pressure is sufficiently maintained to expel product from pouch or bag 3. An air circuit is formed that includes space 105 between the pouch 3 and the canister 2 and that creates a channel allowing air to pass between the walls of the pump and the hole. When button 5 is actuated, channel is open, and it is closed when button is raised. In this case, button 5 functions as a trigger.

The cap $ houses the pump or actuator mechanism 102. Tbe pump mechanism 102 may include an orifice insert, actuator, stem, inner gasket, outer gasket, valve cap, stem, spring cup, spring, valve housing, and dip tube. The pump mechanism 102 includes a valve that allows for 360 ° rotation and can spray at any angle, even when inverted. The valve may be made from chemical resistant material such as polyoxymethylene or other often used plastics. Canister 2 may be fabricated from materials such as polyamide-imide or other materials conventionally used in can linings.

Dispenser device 1 includes an extension applicator arm 7, shown as having a fixed length and in a lowered or storage position.

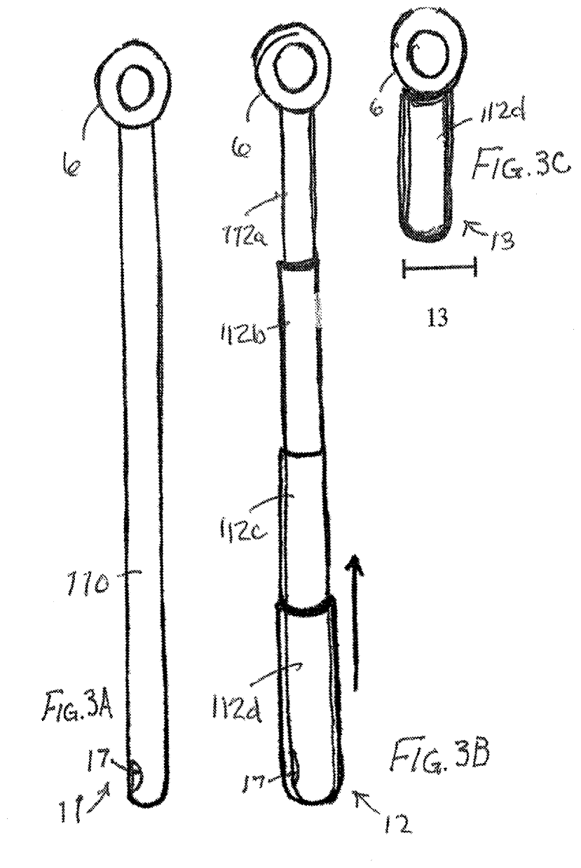

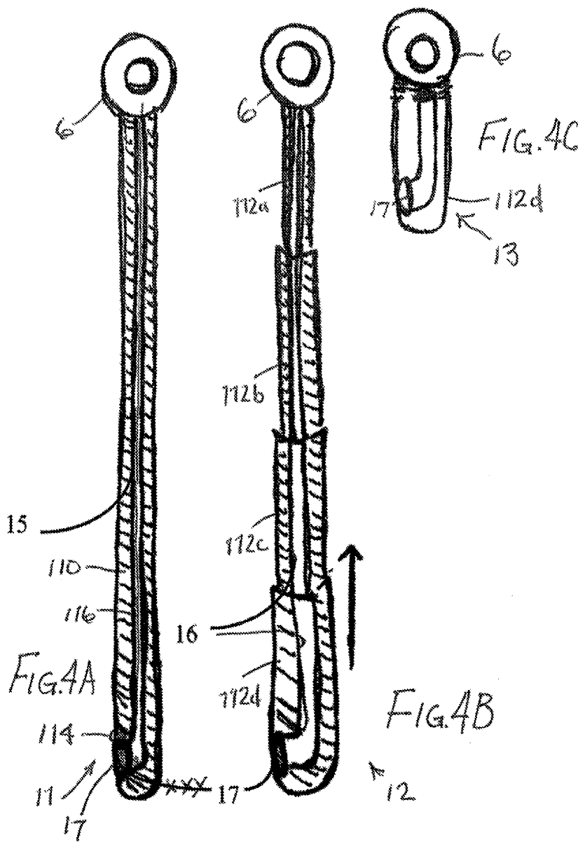

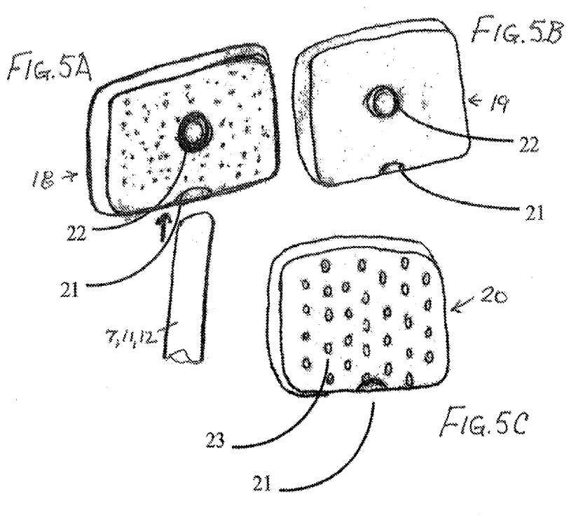

Applicator arm 7 is connected by living hinge 6 to cap 4. Hinge 6 may alternatively take the form of a rotatable ball and joint mechanism. Hinge 6 acts as a universal joint which allows for rotation both horizontally and vertically through a substantial arc approaching 360°. Extension applicator arm 7 ranges in length depending on size of canister 2, but will not exceed the length of the canister, typically ranging from 3 to 10 inches. Variations in the structure of extension applicator arm 7 maintained are depicted in FIGS. 3A-3C and 4A-C. Optional applicator end adaptors are shown in FIGS. 5A-5C.

Applicator arm 7 defines a chamber or conduit 7a extending longitudinally through extension applicator arm 7. A tube 7b extends through chamber or conduit 7a and hinged or rotatable ball joint mechanism 6 and communicates with an opening or nozzle 7c at a free end of applicator arm 7. At an end opposite nozzle 7c, tube 7b is connected to pump or actuator mechanism 102. Tube 7a is configured to carry the liquid fluid, solid (powder), colloidal suspension, or gas from supply canister 2 to nozzle 7c at the free end of extension applicator arm 7.

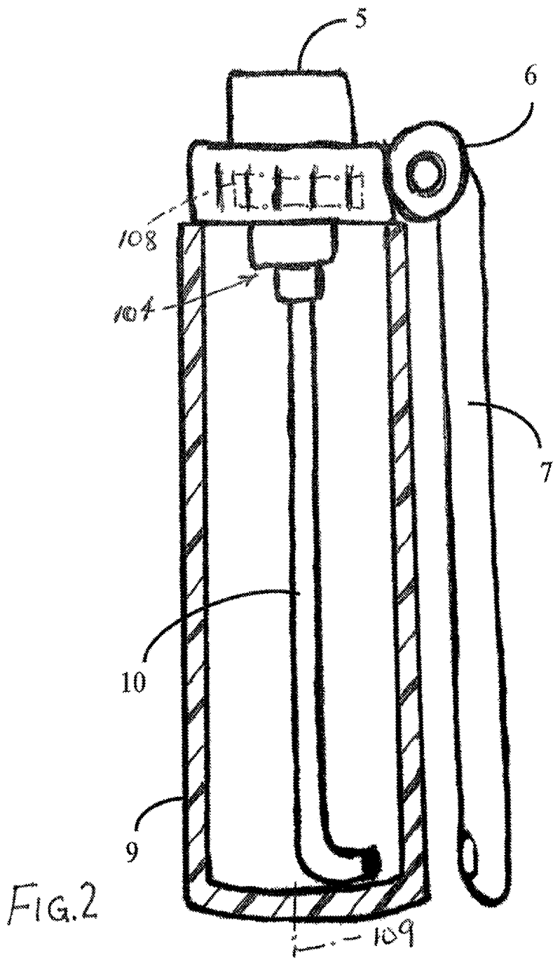

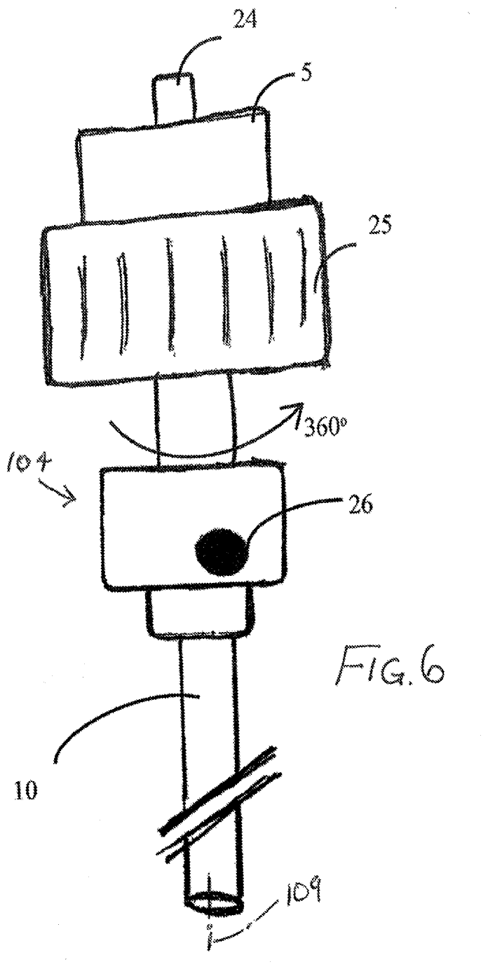

FIG. 2 represents a partially sectional view of an alternative dispenser device 8. A rigid canister body 9 carries a cap 4′ with a pump or actuator 108, a dip tube 10, a hinged or rotatable ball and joint 6′, an extension applicator arm 7′, and a venting system (not shown). The venting system allows air to enter from the ambient environment when a partial vacuum forms as product is expelled during each actuation. A valve assembly 104, which may be included as part of pump or actuator 108, shown on a larger scale in FIG. 6, connects to a cap 25 that carries extension applicator arm 7′. Valve assembly 104 includes a stem valve flange 26 that cooperates with cap 25 to connect to canister body 9 by sandwiching or clamping an upper wall of the canister. Valve assembly 104 houses a ball bearing, including a rod member 24, that allows for 360° rotation about a longitudinal axis 109 of the canister body 9 and the cap 25, allowing the valve to open and the dispenser 8 to spray fluid at any angle, even when inverted. Various components of valve assembly 104, including valve flange 26 may be made from chemical resistant material such as polyoxymethylene or other often used plastics. Canister 9 may be manufactured from materials such as polyamide-imide or other often used can linings.

FIGS. 3A-3C represent two variations 11 and 12 of extension applicator arm 7 (FIGS. 1 and 2). Applicator arm 11 (FIGS. 3A and 4A) is of a fixed length, ranging from 3-10 inches depending on canister size. This arm 11 may be built from firm, chemical-resistant material, such as polyvinyl chloride, polypropylene, polyvinylidene difluoride, and polytetrafluoroethylene. This applicator arm 11 may also be built from more flexible materials such as an impact modified acrylic, polycarbonate, or other suitable ultraviolet resistant, high impact, suitable moldable polymeric plastics materials, as well as of modified rubbers or lattices and other suitable materials. The hinge or ball and joint mechanism is shown at 6. In applicator arm 11, an outer tube 110 surrounds an inner tube 15 used to deliver liquid.

As shown by a comparison of FIGS. 3B and 4B with FIGS. 3C and 4C, extension applicator arm 12 is collapsible from a fully extended configuration (FIGS. 3B and 4B) to a fully collapsed configuration 13 (FIGS. 3C and 4C). More specifically, applicator arm 12 is a telescoping assembly of several telescoping tubular parts 112a, 112b, 112c, 112d connected at one end to hinge or ball and joint mechanism 6 and provided at a free end with a nozzle 17. Nozzle 17 may be modified to change the geometry or shape of the spray plume. Tubular pans 112a, I 12b, 112c, 112d each include an inner tube 16 built from firm, chemical-resistant material, such as polyethylene, polyvinyl chloride, polypropylene, polyvinylidene difluoride, and polytetrafluoroethylene. Alternatively, the inner tube 16 may be built from more flexible materials such as an impact modified acrylic, polycarbonate, or other suitable ultraviolet resistant, high impact, suitable moldable polymeric plastics materials, as well as of modified rubbers or lattices and other suitable materials. The arm 12 may be provided with a sealing member or compound between tubular parts 112a, 112b, 112c, 112d to prevent leaking. Collapsed applicator 12 takes form 13 where all parts 112a, 112b, 112c are contained in the most distal piece 112d.

Alternatively or additionally, extension applicator 11 may be used as a device for phototherapy in which a light source 114 is located at the distal end of the applicator arm, in addition to or instead of nozzle 17 (FIG. 48). The canister 9 in this alternative use would house a power source. A fiber optic light guide 116 extends through the applicator arm 11, for instance, along a wall of inner tube 15. This phototherapy option may be used alone or in conjunction with sprayable media as a co-therapy.

Inner tube 15 and segmented or concatenated inner tube 16 extend through the respective applicator arm 11, 12 and the hinged or rotatable ball joint mechanism 6 to pump or actuator mechanism 102 or 108 on an upstream side and to nozzle 17 on a downstream side. Tubes 15 and 16 are configured to carry the liquid fluid, solid, colloidal suspension, or gas from supply canister 2 or 9 to nozzle 7c or 17 at the free end of extension applicator arm 7 of FIG. 1 or 2, respectively

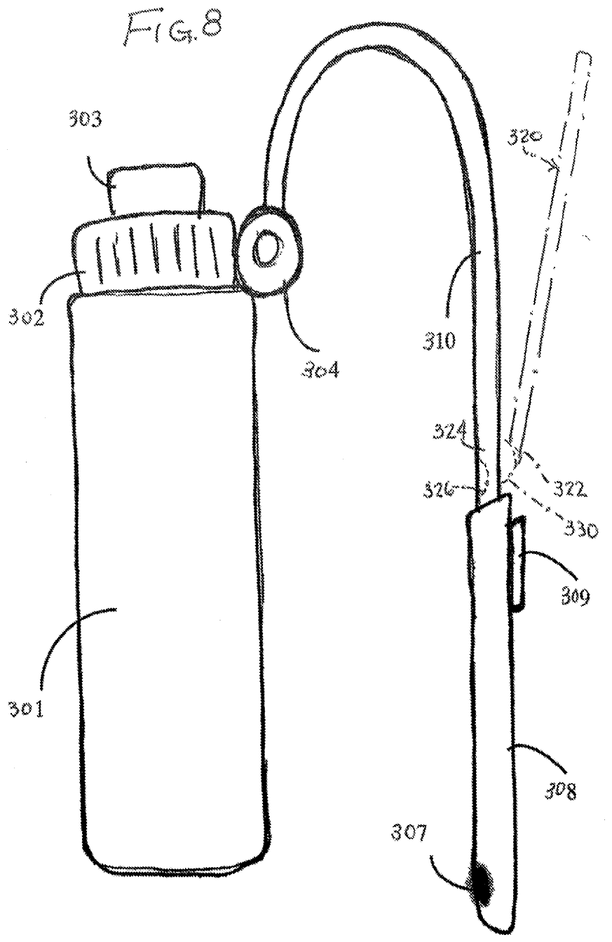

FIGS. 5A-5C depict optional attachments 18, 19, 20 that can be attached to the ends of the extension applicator arms 7, 11, 12 (FIGS. 1, 2, 3A-3C and 4A-4C) and to applicator handpiece 308 (FIG. 8). Each attachment 18, 19, 20 may be disposable. Attachment 18 is a hollow sponge pad made from porous material that allows the user to rub in applied topicals. The free end or tip of the applicator arm 7, 11, 12 is inserted into sponge pad 18 through a slot 21. A hole 22 in sponge pad 18 aligns with the nozzle 17 of the applicator arm 7, 11, 12 so that sprayable media can be dispensed. The attachment 19 is a non-porous pad made from resilient material, for example a nylon or polypropylene exfoliator pad. Alternatively, the attachment 19 may be a UVA, UVB or other wavelength light for optional phototherapy. This may be used alone or in combination with a spray medication that can be dispensed through hole 22. Attachment 20 is a pad formed with an array of holes 23 along the pad to evenly distribute the sprayable liquid.

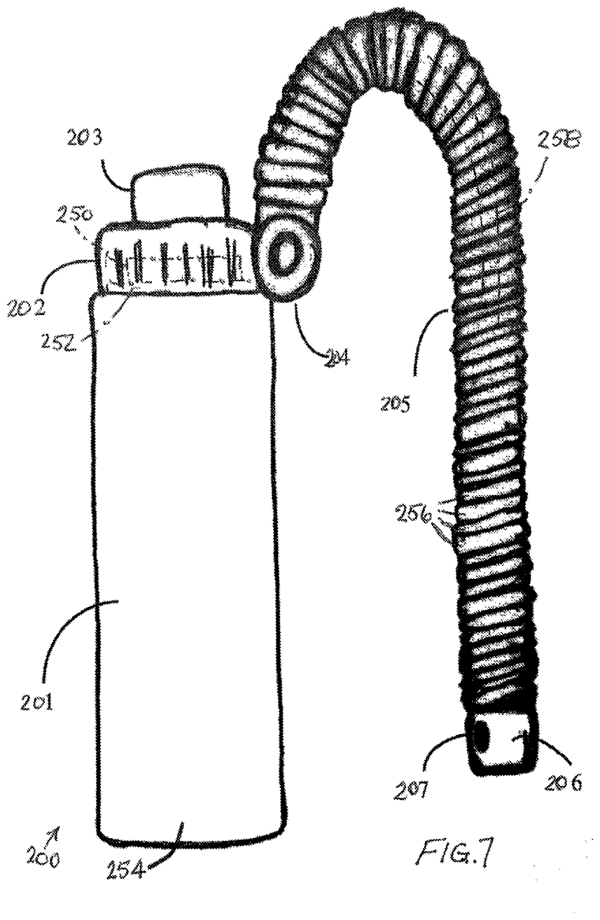

FIG. 7 shows a dispenser device 200 including a canister or rigid container 201 with a pump mechanism 250 mounted on a neck 252 thereof. Pump mechanism 250 is sealed and fixed to the neck 252 within a cap 202. The cap 202 houses the pump mechanism 250, which is operated via a push button 203. The pump mechanism 250 includes an orifice insert, actuator, stem, inner gasket, outer gasket, valve cap, stem, spring cup, spring, valve housing, and dip tube. Pump mechanism 250 includes a rotary valve that allows for 360° rotation about a longitudinal axis 254 of the canister 201 and can spray at any angle, even when inverted. The valve may be made from chemical resistant material such as polyoxymethylene or other often used plastics. Canister 201 may be built from materials such as polyamideimide or other often used can linings.

FIG. 7 depicts an elongate flexible extension or applicator arm 205 that is a malleable, position retaining connector between canister and nozzle. Applicator arm 205 is a jointed or segmented tube wherein individual substantially rigid segments or tubular elements 256 are pivotably connected to one another to enable reconfiguration of the applicator member 205 by the application of manual bending force. Applicator 205 is optionally connected by a living hinge 204 to cap 202. Hinge 204 may alternatively take the form of a rotatable ball and joint (universal) mechanism. Hinge or universal joint 204 allows for rotation both horizontally and vertically through a substantial arc approaching 360°. However, it is to be noted that the desired rotational degrees of freedom may be provided by the flexibility of the applicator member 205 alone.

Applicator arm 205 ranges in length depending on size of canister 201, and where there is a living hinge or ball-and-socket universal joint 204, will typically have a length between about 3 inches and about 10 inches. Alternatively, where there is no hinge or universal joint 204, applicator arm 205 can exceed the length of the canister 201 and ranges from several inches to over thirty inches in length. Flexible applicator arm 205 has a hollow interior so that a tube (not separately shown) can extend from the hinge or universal joint 204 to a terminus nozzle 207 to carry or guide contents from the container or canister 201. Nozzle 207 is used to spray media in a desired spray pattern at a desired location. Nozzle 207 may be provided in a head 204 that is rotatable and may have various attachments as described hereinabove with reference to FIGS. 5A-5C.

Where applicator arm 205 includes, inside the segmented or multiply articulated reconfigurable outer tube, an inner tube 258 that tube may be made of flexible material such as an impact modified acrylic, polycarbonate, or other suitable ultraviolet resistant, high impact, suitable moldable polymeric plastics materials, as well as of modified rubbers or lattices and other suitable materials. The housing of the flexible arm 205 in FIG. 7 may be implemented by having a polymeric tube covering a helical-wire and twined reinforcement.

FIG. 8 shows a fluid dispenser device 300 with a composite extension applicator arm 305 including a flexible tube or hose 310 and a rigid applicator handpiece 308. Tube or hose 310 connects the rigid applicator handpiece 308 to a living hinge or universal ball joint 304 which is attached to a cap 302 on a canister body 301. Flexible tube 310 ranges in length from 12 to 36 inches. Again, owing to the flexibility of the applicator tube or hose 310, the living hinge or rotatable ball joint 304 may be omitted. Applicator handpiece or handle 308 is used to direct the dispensing of contents from the canister 301 and may be between 0.5 and 12 inches in length, where a living hinge or universal ball and socket joint 304 is provided. Alternatively, in the absence of hinge or joint 304, tube or hose 310 may extend up to three feet in length. The tube or hose 10 may be built from firm, chemical resistant material, such as polyethylene, polyvinyl chloride, polypropylene, polyvinylidene difluoride, and polytetrafluoroethylene, lite applicator handpiece or handle 208 may contain an actuator or pushbutton 309, for enabling a dispensing spray of fluidic material from the canister 301 via a nozzle 307 at the end of handpiece or handle 308.

Alternatively, the rigid applicator handpiece 308 may be replaced by a separate rod 320, connected only at a free or distal end 322 to a free end 324 of applicator tube or hose 310 (e.g., via a ball-and-socket universal joint 330), proximately an outlet or nozzle 326 thereof The user grasps a proximal end 328 of the rod 320 and manipulates the rod to direct the free end 324 of the applicator tube or hose 310 to a desired location.

Fluid dispenser device 300 includes a pump or actuator that is the same as any one of pumps or actuators 102, 108, 250. Fluid dispenser device 300 may include a tube performing the function of tube 7b, 11, 15, 258.

Canisters 201 and 301 in FIGS. 7 and 8 may be used as a spray pump, pressurized aerosol, or a bag-on-valve (BOV) system.

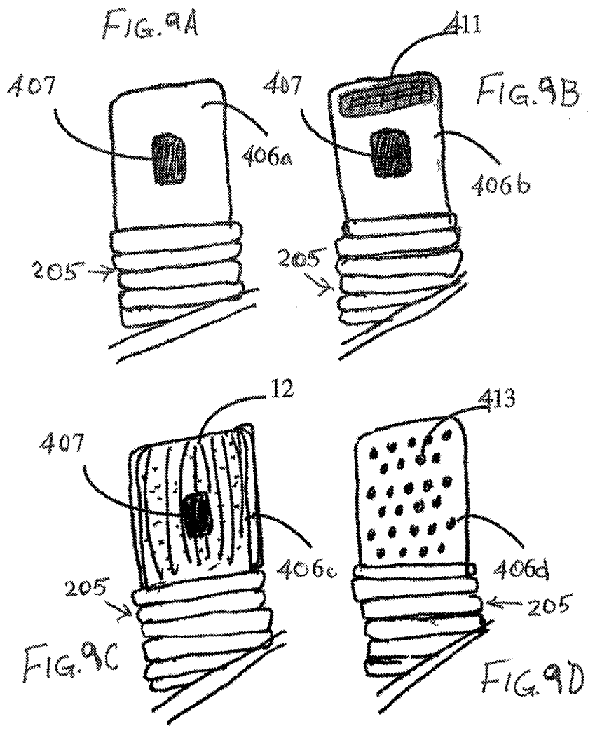

FIGS. 9A-9D illustrate optional attachment heads 406a, 406b, 406c, 406d that can be coupled to the end of the extension applicator arm 205 in FIG. 8, Attachment heads 406a, 406b, 406, 406d each have a nozzle hole 407. Attachment 406b has a source 411 for emitting UVA, UVB or other wavelength light, such as blue light, for optional phototherapy. This may be used alone or in combination with a spray medication that can be dispensed through the hole 407. Attachment head 406c takes the form of a hollow sponge pad made of porous material that allows the user to rub in applied topicals. Alternatively, attachment head 406c may be a non-porous pad made from resilient material, for example a nylon or polypropylene exfoliator pad. Attachment head 406d is formed with a series or array of holes 413 along the pad to evenly distribute liquid spray. The structures of heads 406a, 406b, 406c, 406d may additionally be incorporated as modifications to the extension applicator arms 7, 11, 12, and handpiece 308.

It is to be noted that pump or actuator mechanism 102, 108, 250 may be implemented alternatively by a mechanical pump operated by push button 5, 203, 303, which structure well known in the dispensing arts. This alternative avoid the release of hydrocarbons into the atmosphere and facilitates manufacture, transport and distribution.

### EXAMPLES

The present invention provides a device that may be used to apply any sprayable media or fluidic composition, these terms referring to any fluid, solid, colloidal suspension, or gas capable of being sprayed.

Sprayable media may contain one or more of the following: active ingredients, permeation enhancers, film formers plasticizers.

The term “active ingredient” refers to a mixture constituent intended to be absorbed through the skin. Active ingredients include drugs, cleansing agents, drying agents, and cosmetics. Further examples are presented below.

The word “solubilizer” refers to a constituent that aids in the dissolution of an active ingredient as a solid, liquid, or gas. Some examples of solubilizers include propylene glycol monocaprylate, diethylene glycol monoethyl ether, acrylate and methacrylate ester polymers/copolymers, surfacants, polyhydric alcohols, D-a-Tocopheryl polyethylene glycol 1000 succinate (Vitamin E TPGS), and Vitamin E.

As used herein, the term “permeation enhancer” refers to a constituent that is meant to enhance skin penetration of an intended active ingredient . These constituents may also be referred to as “ accelerants” or “sorption promoters”. Examples include DMSO, ureal, propylene glycol, alcohol (such as ethanol), oleic acid, sodium lauryl sulfate, and fatty acid esters.

The term “film-formers” is used herein to denote agents that are used to leave a flexible, elastic, and cohesive covering over the skin. Examples of such agents are acrylates arylamides and polyvinylpyrrolidone.

Plasticizers are agents that aid in a composition forming of the film. Some examples include propylene glycol, dimethyl isosorbide, tritheyl citrate, castor oil, and polyethylene glycol.

An extension applicator arm as described herein may be used to apply a spray, either pump or pressurized, containing an active ingredient, vehicle or solubilizer, permeation enhancer, film-former, plasticizer, and propellant if necessary for pressurized systems. Any combination of the above mentioned components might be used. The preferred application of mixtures to be applied to the skin is via a metered dose over a desired surface area.

The present invention contemplates a method of applying a topical, pharmaceutical spray composition containing a drug or combination of drugs as solution or suspension in a vehicle. The exact formulation will vary depending on medicament used and desired release profile.

A drug includes, but is not limited to, anti-acne agents, topical anesthetics, anti-infectives, anti-rosacea agents, antibiotics, antifungals, antihistamines, antineoplastics, antipsoriatics, antivirals, astringents, debriding agents, depigmenting agents, emollients, keratolytics, non-steroidal anti-inflammatories, photochemotherapies, rubefacients, steroids, vaginal treatments, and any combination of the above-mentioned therapies.

Drugs listed are to be applied topically. Examples of anti-acne agents are erythromycin, clindamycin, minocycline, and tetracycline, benzoyl peroxide, salicylic acid, and retinoids; examples of topical anesthetics are lidocaine, epinephrine tetracaine, benzocaine, and Lidoderm. Examples of anti-infectives include malathion, crotamiton, sinecatechins, benzyl benzoate, imiquimod, benzyl alcohol, boric acid, and chloroxine; examples of anti-rosacea agents include azelaic acid, metronidazole, and brimonidine. Examples of antibiotics include sulfacetamide sodium, silver sulfadiazine; erythromycin, bacitracin neomycin, and tetracycline; examples of antifungals include benzoic acid, clioquinol, econazole, oxiconazole, nystatin, terbinafine, and sodium thiosulfate. Examples of antihistamines include caiamine, diphenhydramine, and doxepin; examples of antineoplastics include fluorouracil, imiquimod, ingenol, and mechlorethamine. Examples of antipsoratics include tazarotene, betamethasone, calcipotriene, calcitriol, anthralin, clobetasol, methoxsalen, and resorcinol; examples of antivirals include penciclovir and acyclovir; examples of astringents include acetylcysteine, witch hazel, zinc sulfate. Examples of debriding agents include balsam peru, castor oil, typsin, papain, chlorophyllin copper, urea, and collagenase. Examples of depigmenting agents include flucinolone, hydroquinone, tretinoin, and hydroquinone. Examples of emollients include salicylic acid, vitamin A, vitamin D, urea, ammonium lactate, ammonium lactate, pramoxine, hydrocortisone, lactic acid, and petrolatum. Examples of keratolytics include salicylic acid, podofilox, and podophyllum. Examples of non-steroidal anti-inflammatory agents include diclofenac and capsaicin, Examples of photochemotherapeutics include aminolevulinic acid, methoxsalen, and methyl aminolevulinate. Examples of rubefacient agents include menthol, methyl salicylate, trolamine salicylate, camphor, and capsaicin. Examples of steroids are amcinonide, diflorasone, clocortolone, fluticasone, ammonium lactate, clobetasol, betamethasone, flurandrenolide, desonide, desoximetasone, hydrocortisone, and triamcinolone; examples of vaginal treatments include clortrimazole, butoconazole, hydroxyquinoline, terconuole, miconazole, and sulfanilamide.

The disclosed invention includes a method of applying an insect repellent composition to human skin in a solution or suspension. Examples of insect repellent include N,N-diethyl-in-toluamide (DEET), picaridin, and IR3535 (Ethyl butylacetylaminopropionate).

The present invention further includes a method of applying cleansing agents, protective agents, anti-itch, and skin drying agents. Examples of cleansing agents include soaps, shampoos, and antiseptic skin cleansers (i.e. chlorhexidine gluconate). Examples of protective agents include oil suspensions, powder suspensions, and sunscreens. Examples of anti-itch agents that are OTC are chamomile, eucalyptus, camphor, methanol, zinc oxide, talc, glycerin, and calamine. Examples of drying agents include talcum powder suspensions, deodorants, aluminum salt solutions, and aluminum acetate.

The present invention contemplates a method of applying cosmetics to the body. Examples of cosmetics include concealers, perfumes, fragrances, bronzers, sun-less tanners, sunscreens, moisturizers, and oils.

The present invention may be used with a lightsource for phorotherapy. Examples include UVA (315-400 nm) to treat vitiligo, psoriasis, eczema, solar urticarial, graft-vs-host disease, and mycosis fungoides; UVB (280-315 nm) is used to treat psoriasis, eczema, pruritus, parapsoriasis, as well as other skin diseases. This may be used singularly as a phototherapy or in combination with a topical treatment.

### Case Examples of Present Invention

Presented here are three cases where the invention for the extension applicator may be well equipped:

### Example Case 1—Psoriasis Topical Therapy

i. Problem To Be Solved: Moderate to severe plaque psoriasis (MSPP) is a systemic inflammatory autoimmune disease that affects approximately 2% of the global population, and over 7 million American adults annually.^{1, 2, 3} The pathophysiology of MSPP is readily visible, typified by irritating and itchy skin lesions that most commonly occur on the scalp, limbs, and trunk. The skin lesions can cause extreme physical and psychological discomfort, and have a profoundly negative impact on quality of life. The combined treatment and productivity costs associated with psoriasis in the US each year are estimated to be $112 billion.⁴ While systemic therapies are available, 80% of patients are treated with prescription topical drug products, as these therapies are associated with less frequent and severe side effects.⁵ Lesions presenting on the harder-to reach areas of the trunk, or lower extremities are common and present a unique treatment challenge because of the large surface area involved and difficulty in self-applying topical treatments to the hard-to-reach areas (FIG. 1A). This problem is exacerbated in patient groups who live alone or have physical limitations. These groups include singles, patients with physical handicaps, the elderly, and those with limited mobility. Limited mobility is common in the psoriatic patient population, with 30% of patients suffering from psoriatic arthritis (PsA).⁶ The present invention facilitates the self-application of topical treatments for MSPP to the harder-to-mach areas.

ii. Current Paradigm: Clobetasol propionate is a corticosteroid and is widely prescribed for the effective relief of itching, flaking, and scaling related to MSPP. Clobex® (Galderma Laboratories Submission Control No: 155134) is the only topical. spray product available for the treatment of MSPP and was originally developed to expand upon treatment options for patient groups experiencing difficulties adequately treating their psoriasis.⁷ Available as a 0.05% clobetasol propionate solution that is dispensed through a small adjustable nozzle, Clobex® remains the category leader for MSPP. The nozzle is designed to facilitate application of product to the target area on the scalp and elbows, but patients report difficulty applying the spray to harder-to-reach areas such as the back limits its effectiveness.^{8, 9, 10, 11} There remains an unmet need for the device of the present invention, which dramatically improves the patient's ability to self-apply topical formula and provide a much needed alternative to Clobex®.

iii. Product: A 0.05% clobetasol propionate spray as a bioequivalent topical drug product for Clobex®. This product is unique from all approved topical drug products in utilizing the present dispenser device with extension applicator arm 7, 11, 12, 205, 310/308 to facilitate the self-application of the drug product to any area of the body, including the harder-to-reach areas of the back, shoulders, and lower extremities. The device as described herein is a proprietary lightweight plastic device that is permanently affixed to the top of the filled aerosol or non-aerosol can (200 mL) during manufacture, the entire product intended for sale as a disposable unit. A 360° dose-metered valve is utilized to enable dose-metered spray at any angle, even when inverted. The applicator facilitates the dose-metered self-application of the bioequivalent Clobex® formula to areas of the body that might otherwise not be accessible with a traditional spray product. Submission of a 505(b)(2) New Drug Application (NDA) to the FDA is contemplated, which permits approval of applications that rely partially on literature or on an Agency finding of safety and/or effectiveness for an approved drug product.¹² It is expected that this pathway will allow for a cost effective and time efficient route to approval. The studies in this Phase I SBER proposal aim to demonstrate in vitro BE and satisfy requirements for a pre-IND meeting with the FDA.¹³

iv. Value of the Solution: MSPP affects between 1.5% to 5% of the population in developed countries.¹⁴ In the US alone, where approximately 7 million adults are affected by MSPP, the direct and indirect costs associated with treatment and productivity loss is estimated to exceed $112 billion per year.⁴ MSPP is associated with depression, immune-mediated inflammatory conditions; and obesity. The psychological impacts of MSPP can be profound, leading to negative impacts on quality of life that include higher rates of unemployment or missed time at work, behavioral and emotional difficulties, and a negative effect on performance at work/school.¹⁵ Clobex® is currently the only available clobetasol propionate spray, and was originally developed to expand upon treatment options for patient groups experiencing difficulties adequately treating their psoriasis including: singles, patients with physical handicaps, and elderly patients. However, a majority of patients still report difficulties applying the Clobex® product to the harder-to-reach areas of the shoulders, back, and lower extremities. There remains a significant need to expand on the armamentarium of available topical spray products, thereby presenting patients with a multitude of treatment options and driving down the cost of the drug product.

v. Technological Innovation: It is contemplated to use the adjustable applicator of the present invention to enable the self-application of drug product, including a bioequivalent clobetasol propionate spray, to a user's entire body, including the harder-to-reach areas of the shoulders, back, and lower extremities. The extension applicator arm 7, 11, 12, 205, 310/308 is permanently affixed onto the filled canister 2, 9, 201, 301 during manufacture, and the entire product is sold as a disposable unit. As an example, in situations where the user would like to spray his or her back, he or she may hold the applicator in an inverted position over their shoulder, and direct spray toward the back, thereby reaching areas that might otherwise not be accessible. The applicator comprises an arm 7, 11, 12, 205, 310/308 with an internal plastic tube 7b, 15, 16, 258, a nozzle 7c, 17, 207, 307, a pump or actuator mechanism (including a valve) 102, 108, 208, a tube opening, an actuator 5, 203, 303 such as a posh button or toggle switch, and a clamp or cap 4, 202, 302. The extension applicator arm 7, 11, 12, 205, 310/308 houses the entire length of the tube 7b, 15, 16, 258, which connects the supply canister 2, 9, 201, 301, containing the bioequivalent formula to the nozzle 7c, 17, 207, 307 at the arm terminus. The pump or actuator (with valve) 102, 108, 250 controls the flow of liquid to the nozzle 7c, 17, 207, 307, and the clamp or cap 4, 202, 302 secures the applicator arm 7, 11, 12, 205, 310/308 directly to the supply canister 2, 9, 201, 301. Pressing the toggle switch or push button 5, 203, 303 releases pressure from the canister 2, 9, 201, 301 and dispenses the bioequivalent formula through the length of the tube 7b, 15, 16, 258 where it is dispensed as a spray through the nozzle 7c, 17, 207, 307. The applicator arm 7, 11, 12, 205, 310/308, constructed from plastic and weighing only about 26 grams, is designed to be lightweight and ergonomic. It provides accessibility to locations on the body that may be difficult to reach, such as the length of the users shoulders and back. Unlike commercial products that contain a nozzle located immediately atop a supply canister, a dispensing device in accordance with the present invention maintains a distance between the nozzle 7c, 17, 207, 307 and supply canister 2, 9, 201, 301to facilitate a dose-metered spray to any area of the body,

vi. Potential Impact: Clobex® is the only clobetasol spray product available, but self-application to the back, shoulders, and lower extremities remains challenging.¹¹ The present delivery device will facilitate self-application of the Clobext® bioequivalent to all areas of the body and address a significant unmet health and commercial need, especially the greater than 50% of psoriasis patients suffering from joint stiffness and pain in addition to the 30% diagnosed with PsA.⁶

vii. Rationale for the preliminary studies: The primary objective of the preliminary studies was to de-risk the bioequivalent drug product for more cost-intensive Phase I SBIR studies. Exhaustive diligence was performed to determine what would be required in developing a bioequivalent formulation that would be compatible with the drug delivery device of the present invention.

viii. Summary of the established safety and efficacy data relating to the reference listed drug: Clobex®0 was approved in its spray formulation in 2005, with the patent set to expire Mar. 24, 2018.^{16, 17} The efficacy of Clobex® spray has been evaluated in two clinical trials. In a double-blind, randomized, vehicle-controlled clinical trial of 27 patients with MSPP, after 4 weeks of twice daily treatment, 100% of patients reported lesions as “none” or “mild” in comparison to 28% of those treated with the vehicle.¹⁸ In a second double-blind, randomized clinical trial of 120 patients with MSPP. after 4 weeks of twice daily application of clobetasol propionate spray, 78% of patients in the treatment group reported clear or almost clear skin, compared to only 3% of the control group.¹⁹ The results of these studies reveal clobetasol propionate spray as a highly effective and well-tolerated form of topical treatment.

IX. Consumer Product Research: Consumer feedback is an important metric for designing products that will be commercially viable, and well received. To assess the consumer perception and purchase intent of the psoriasis product, published literature was reviewed. Clobetasol propionate is the most commonly used MSPP topical treatment agent used in the US.²⁰

Results: in an analysis of 10 randomized studies evaluating each vehicle form of clobetasol propionate, the spray formulation achieved the highest percentage of clearance of lesions compared to other formulations.²¹ This result could be related to greater patient adherence of the spray formulations that are “easier-to-use” and “lighter.”²² Spray formulation is considered especially valuable for those suffering from psoriatic lesions on larger surface areas of the body (>15-20%) and regions that are difficult-to-reach.²¹

x. Impact on Quality of Life: Patients plagued with psoriasis can experience significant anguish and suffering negatively impacting their quality of life (QOL).²³ Results: in a sample set of 2,236 patients from 5 controlled clinical trials, QOL analysis measured by the Dermatology Life Quality Index (DLQI) and the Koo-Menter Psoriasis Index identified the clobetasol propionate spray as the best formulation for improving QOL. When used twice a day for 4 weeks, 80% of patients reported significant improvement in a number of aspects of QOL. This study, however, only addressed patients suffering from plaque psoriasis of the scalp and did not include patients who suffer from the disease in more difficult to reach areas of their body, such as trunk and limbs. These results suggest that increasing the ability to apply topical therapies to other areas will significantly increase the QOL for all patients suffering from MSPP.

xi. Patient Acceptance: Critical to the success of psoriasis management is adherence to treatment regimens, where first-line therapy involves the use of topicals. A survey was presented to an expert panel including members of the psoriasis group of the Spanish Academy of Dermatology and Venereology, designed to evaluate adherence to topical therapies in the psoriatic patient population.²⁴

xiii. Results: Based on survey responses, guidelines for improving psoriatic patient treatment adherence were proposed and these include: 1. Greater interaction between patient and health care provider; 2. Provide better information on the nature of the disease; 3. Simplify the therapy; 4. Give written instructions and physician or nurse contact information; 5. Deliver evidence-based therapies; 6. Prescribe products that are easy and convenient to apply; and 7. Ensure patient satisfaction with vehicle. The present delivery device satisfies a number of these guidelines, suggesting that the PHD bioequivalent drug product will be well received in the marketplace. Additional data related to viability metrics, pricing, demographics of likely customers, and open feedback was also collected. Importantly, Key Opinion Leaders (KOL) assert that the PHD product will satisfy an unmet commercial and health need, especially in populations with limited mobility and lesions in difficult-to-reach areas.

xiii. Formula Attributes: The psoriatic product contemplated for application with the dispensing device of the present invention is a 0.05% clobetasol propionate (w/w) formula designed to be delivered as a spray and demonstrate bioequivalence with the corresponding Clobex® product. It can be sprayed on exposed skin to relieve itching, scaling and flaking associated with psoriasis.

### Sample Formulation (% w/w) of RLD

0.05% clobetasol propionate

49.3% alcohol

0.3% isopropyl myristate

0.1% sodium laurel sulphate

0.1% undecylenic acid

### Example Case 2—Acne Topical Therapy

Problem To Be Solved: Acne vulgaris (acne), a chronic inflammatory disease of the pilosebaceous unit, is one of the top 10 most prevalent diseases, globally [7] and is the most common skin condition in the USA, affecting 40-50 million Americans each year. [1] Acne occurs most commonly in adolescents, with approximately 85% of people aged 12-24 experiencing at least a mild form of the disease. [1] The pathophysiology of acne is readily visible, typified by comedones, pimples, and cysts that occur on the face, neck, chest, back, shoulders, and upper anus. [2] Effective treatment of patients with acne is important because the adverse affect on physical appearance can lead to issues with self-esteem and psychological wellbeing. [1] The psychological effects of acne have a significant impact on the US healthcare system, and are estimated to be responsible for annual losses related to treatment and decreased productivity of $3 billion. The treatment of acne most commonly involves over-the-counter (OTC) or prescription topical therapies. [8, 9] Back acne occurs in 61% of patients [3, 4] and presents a unique challenge because of the large surface area involved and difficulty in applying topical treatments to hard-to-reach areas. [3] While the treatment of facial acne has been reported on extensively in the literature, truncal acne, including the back and shoulders, has received considerably less attention. [3] This is surprising, given the large number of patients that present with truncal acne, and their desire for treatment. [3, 10] PHD Biosciences has developed a benzoyl peroxide (BPO) based topical acne spray product that utilizes a proprietary extension applicator to facilitate the homogenous self-application of the treatment formula to the hard-to-reach areas of the back and shoulders.

ii. Current Paradigm: While topical acne treatments are widely available, they are challenging to apply to the harder to reach areas of the back and shoulders. [3] There remains a need for a new type of topical acne product that can facilitate homogeneous self-application of treatment formula to the harder to reach areas of the body. Consumer market research (see Preliminary Results) and discussions with clinical dermatologists and customers support the hypothesis that the PHD acne spray product proposed in this Phase I SBIR application will be widely adopted by the global market and address an urgent public health need.

iii. Product: A proprietary over-the-counter (OTC) aerosol spray product for body acne has been developed that conforms to all rules set forth in the Food and Drug Administration (FDA) guidelines for topical OTC acne drug products [11], and contains benzoyl peroxide (BPO) as the active acne ingredient. The product will be marketed as a twice-daily treatment for truncal acne and is unique from all commercial acne products because it utilizes the present dispensing device with its extension applicator arm to enable the homogenous self-application of the acne treatment formula to any area of the body. As discussed hereinabove, the applicator is a proprietary lightweight plastic device that is permanently affixed to the top of the filled aerosol can (200 ml) during manufacture, and the entire product being marketable as a disposable unit. The product incorporates a 360° valve that enables continuous spray at any angle, even when inverted. The applicator arm 7, 11, 12, 205, 310/308 facilitates the homogenous self-application of the acne treatment formula to areas of the body that might otherwise not be accessible with a traditional spray product. The Food and Drug Administration (FDA) regulates OTC topical acne drug products, and the commercialization of these products requires compliance with guidelines set forth in the agency's published Final Rule. [12, 13] Experiments in this Phase I SBIR proposal aim to demonstrate the safety and efficacy of the product in human clinical studies, and are necessary to advance the product towards commercialization.

iv. Path to Market Penetration. It is contemplated that the present dispensing device with its various alternative payloads can be distributed through an existing network that sells sunscreen and self-tanning products in twenty-five countries. This existing distribution network will be exploited to commercialize the new acne product desciibed in this Phase I SBIR. The brand has a line of aerosol sunscreen and self-tanning products that utilize the present dispensing device with its extension application arm 7, 11, 12, 205, 310/308, and consumer acceptance of this device has been validated through commercial sales of these sunscreen and self-tanning products. The acne product described in this proposal is unique from all commercial products.

v. Value of the Solution: The Global Burden of Skin Disease Study 2010 estimated that there are over 650 million sufferers of acne worldwide, with 40-50 million of those sufferers in the US. [1] In the US alone, the direct and indirect costs associated with treatment and productivity loss is estimated to exceed 53 billion per year. From a social perspective it has been found that acne has several negative impacts on quality of life including higher rates of unemployment, anger, behavioral and emotional difficulties, and a negative affect on perfbmiance at work/school. [1] It is anticipated that use of the PHD acne product to treat truncal acne will improve treatment outcomes and contribute to improved quality of life and psychological wellbeing.

vi. Technological Innovation: The body acne aerosol spray product is applied via the adjustable aerosol applicator of the present invention, which enables homogenous self-application by users to their entire body. The applicator arm 7, 11, 12, 205, 310/308 is permanently affixed onto the Idled can 2, 9, 201, 301 during manufacture, and the entire product is sold as a disposable unit. As an example, in situations where the user would like to spray his or her back, he or she may hold the applicator in an inverted position over the shoulder, and direct the spray toward the back, thereby reaching areas that might otherwise not be accessible. The applicator comprises an arm 7, 11, 12, 205, 310/308 with an internal plastic tube 7b, 15, 16, 258, a nozzle 7c, 17, 207, 307, a pump or actuator (with valve) 102, 108, 208, a tube opening, an actuator 5, 203, 303 such as a push button or toggle switch, and a clamp or cap 4, 202, 302. The extension applicator arm 7, 11, 12, 205, 310/308 houses the entire length of the tube 7b, 15, 16, 258, which connects the supply canister 2, 9, 201, 301, containing the bioequivalent formula, to the nozzle 7c, 17, 207, 307 at the arm terminus. The or actuator (with valve) 102, 108, 250 controls the flow of liquid to the nozzle 7c, 107, 207, 307, and the clamp or cup 4, 202, 302 secures the applicator arm 7, 11, 12, 205, 310/308 directly to the supply canister 2, 9, 201, 301. Pressing the toggle switch or push button 5, 203, 303 releases pressure from the canister 2, 9, 201, 301 and dispenses the bioequivalent formula through the length of the tube 7b, 15, 16, 258 where it is dispensed as a spray through the nozzle 7c, 17, 207, 307. The applicator arm 7, 11, 12, 205, 310/308, constructed from plastic and weighing only about 26 grams, is designed to be lightweight and ergonomic. It provides accessibility to locations on the body that may be difficult to reach, such as the length of the users shoulders and back. Unlike commercial products that contain a nozzle located immediately atop a supply canister, a dispensing device in accordance with the present invention maintains a distance between the nozzle 7c, 17, 207, 307 and supply canister 2, 9, 201, 301 to facilitate a dose-metered spray to any area of the body.

vii. Potential Impact: Body acne products are widely available in a variety of dosage forms, but self-application to the shoulders and back remains challenging. [3] The present dispenser/applicator de vice will facilitate even self-application to all areas of the body and address a significant unmet health and commercial need.

viii. Rationale for the preliminary studies: The primary objectives of the preliminary studies were to de-risk the acne product for more cost-intensive Phase I SBIR studies with human subjects. Preliminary investigations were performed in compliance with the animal-use policies of prospective customers in mind. [5, 6] Leading global retailers in the household and personal products industry maintain strict policies that prohibit the purchase of products that have been tested on non-human animals. No animal products were used (animal skins, organs, etc.), or animal testing undertaken in any of the preliminary investigations.

ix. Consumer Product Research: Consumer feedback is an important metric for designing products that will be commercially viable, and well received.

To assess the consumer perception and purchase intent of the current acne product, a product viability test was undertaken by Hit Laboratories (Seattle, Wash.). In this study, 439 domestic participants from an online respondent database (5% confidence interval, 95% level) were presented with information about the product including: digital product images, a short instructional video, and a questionnaire. In this study, “51% of respondents indicated they would prefer to purchase the product or something similar from a local store an unamally high number, suggesting suitability as a retail product.” Respondents with purchase intent ranked the products features in order of importance to them, with the present di s p enseri applicator being “the most imporiani reason to purchase the product over similar products in the category”.

Formula Attributes: The present aqueous acne treatment formula contains a proprietary combination of globally approved and commercially available anti-microbial and anti-inflammatory cosmetic ingredients that are synergistic with benzoyl peroxide (BPO), an FDA approved and generally recognized as safe and effective (GRASE) active ingredient. The formula was designed using Proactiv.™ (category leader) as a benchmark, and is engineered to allow BPO to be effectively formulated at 2.5%, the lowest permissible concentration as defined in the Final Monograph [12], while minimizing the likelihood for irritation. The cosmetic active ingredients are, incorporated to reduce the likelihood for irritation. [16] The formula is readily prepared on multi-kilogram scale at room temperature, using a Silverson L5M-A Mixer, equipped with a stainless steel U-shaped mixing paddle. After mixing for 30 minutes, 140 g the aqueous formula is transferred to a 200 ml polyamide imide lined aluminum can, sealed, and filled with 42 g of a mixture of butane isobutane from Aeropres (Hillsborough, N.J.). The aerosolized formula is delivered to the skin as a fine mist, which dries within 1-2 minutes. The formula has passed thermal bulk and in-package stability studies, and will have a minimum durability period of about 36 months.

### Sample Formulation (% w/vv)

benzoyl peroxide 2.5 %
water
sodium cocoyl isethionate
glycerin, polyethylene
lauryl myristyl benzoate
linoleic acid
linolenic acid
butylene glycol
dimethicone
ethylhexylglycerin
acrylates/C10-30 alkyl acrylate crosspolymer
glycolic acid
sodium hydroxide
phenoxyethanol
fragrance

### Example Case 3—Insect Repellent

i. Problem to Be Solved: The attraction of mosquitos to human hosts is primarily olfactory-mediated and topically applied insect repellents play a crucial role in protecting humans from mosquito bites. A drawback of commercial insect repellent products is that they are challenging to apply to the harder-to-reach areas of the back, legs and feet. Difficulty associated with self-application a topical derniatological products has been noted in a number of disease indications^{25, 26} and is of particular relevance to mosquito repellents because mosquitos are able to detect and feed on unprotected areas of skin as small as 2.5 cm².²⁷ This problem is exacerbated in pregnant women, geriatrics, and the obese; here limited mobility can render self-application of topical agents to the harder to reach areas a unique challenge. The rapid spread of Zika, a flavivirus primarily transmitted by Aedes mosquitos, has highlighted a need for practical solutions to enable facile self-application of insect repellents in populations of limited mobility. Zika remained in obscurity until recently, when it was introduced to Brazil from French Polynesia and spread rapidly through the Americas.²⁸ Originally, Zika disease was characterized as a mild febrile illness, which lasts for up to a week, and presents with symptoms including skin rash, conjunctivnis, muscle and joint pain, and malaise or headache.²⁹ However, it has been determined that Zika infection during pregnancy can lead to microcephaly, making it the first virus in over 50 years discovered to cause birth defects.^{28, 30, 31, 32} In an outbreak of Zika in Micronesia the virus was also been linked to development of the severe neurological disorder, Guillain-Barre syndrome.³³ There is no vaccine to prevent Zika, and the Centers for Disease Control and Prevention (CDC), as part of its Zika prevention strategy, recommends that insect repellent be applied to all exposed skin to avoid mosquito bites.³⁴

ii. Current Paradigm: While CDC recommended topical insect repellents are widely available, there are no products that are designed to facilitate application to exposed skin on the harder to reach areas of the body. Given the rapid spread of Zika in the Americas, there remains an urgent need for novel products that can improve protection by facilitating adequate application of repellent products. Consumer product research (see Preliminary Results) and discussions with dermatologists and industry experts support the hypothesis that the insect repellent product described in this Phase I SBIR application will be widely adopted by the global market and address an existing unmet public health need.

iii. Product Being Developed: Thus, a novel aerosol insect repellent product has been developed that utilizes an Environmental Protection Agency (EPA) registered formula containing 25% N,N-Diethyl-meta-toluamide (DEET) as the active repellent ingredient. The product is unique from all commercial insect repellents because of the present dispensing device with its long extension applicator arm 7, 11, 12, 205, 310/308 to enable the self-application of the repellent formula to any area of the body. The dispenser/applicator is a lightweight plastic device that is permanently affixed to the top of the filled aerosol canister 2, 9, 201, 301 (200 mL) during manufacture, and the entire product is marketable as a disposable unit. A 360° valve is utilized to enable continuous spray at any angle, even when inverted. The applicator arm 7, 11, 12, 205, 310/308 facilitates the adequate self-application of the insect repellent formula to areas of the body that might otherwise not be accessible with a traditional spray product.

iv. Value of the Solution: The rapid spread of Zika has emphasized a need for a comprehensive approach to mitigate the spread of infection. Zika infection can have devastating effects on prenatal development (microcephaly) and has also been linked to Guillain-Barre syndrome.^{30 33} There is no vaccine to prevent Zika, and the CDC recommends that insect repellent be applied to all exposed skin. DEET based repellent products are the most widely utilized, but suffer from a major drawback in that they are not easily self-applied to the harder to reach areas of the body and are only effective where they are applied. The PHD product will improve the consumer's ability to adequately self-apply insect repellent to all areas of the body. The practical and ergonomic design of the present disepenser/applicator will lead to a decrease in the frequency of infectious bites, reducing the transmittance of Zika and other mosquito-borne disease. While it is expected that the product will be widely adopted by the general marketplace, it is anticipated that the product will be particularly useful for pregnant women and other populations with limited mobility.

v. Technological Innovation: The insect repellent aerosol spray product may be self-applied by the user to the entire body, using the adjustable dispensing device with extension applicator arm 7, 11, 12, 205, 310/308. The applicator is permanently affixed onto the filled can during manufacture, and the entire product will be sold as a disposable unit. As an example, in situations where the user would like to spray their back, they may hold the applicator in an inverted position over their shoulder, and direct it toward their back, thereby reaching areas that might otherwise not be accessible with traditional spray products. The applicator comprises an arm 7, 11, 12, 205, 310/308 with an internal plastic tube 7b, 15, 10, 258, a nozzle 7c, 17, 207, 307, a pump or actuator (with valve) 102, 108, 208, a tube opening, an actuator 5, 203, 303 such as a push button or toggle switch, and a clamp or cap 4, 202, 302. The extension applicator arm 7, 11, 12, 205, 310/301 houses the entire length of the tube 7b, 15, 16, 258, which connects the supply canister 2, 9, 201, 301, containing the bioequivalent formula, to the nozzle 7c, 17, 207, 307 at the arm terminus. The pump or actuator (with valve) 102, 108, 250 controls the flow of liquid to the nozzle 7c, 207, 307, and the clamp or cap 4, 202, 302 secures the applicator arm 7, 11, 12, 205, 310/308 directly to the supply canister 2, 9, 201, 301. Pressing the toggle switch or push button 5, 203, 303 releases pressure from the canister 2, 9, 201, 301 and dispenses the bioequivalet formula through the length of the tube 7b, 15, 16, 258 where it is dispensed as a spray through the nozzle 7c, 17, 207, 307. The applicator arm 7, 11, 12, 305, 310/308, constructed from plastic and weighing only about 26 grams, is designed to be lightweight and ergonomic. It provides accessibility to locations on the body that may be difficult to reach, such as the lemtth of the users shoulders and back. Unlike commercial products that contain a nozzle located immediately atop a supply canister, a dispensing device in accordance with the present invention maintains a distance between the nozzle 7c, 17, 207, 307 and supply canister 2, 9, 201, 301 to facilitate a dose-metered spray to any area of the body.

vi. Potential Impact: Topical insect repellent products are widely available in a variety of dosage forms, but adequate self-application remains challenging.²⁵ The insect repellent product discussed herein will mitigate the risk for mosquito borne illness and address a significant unmet health and commercial need.

vii. Rationale for the Preliminary Studies: The primary objective of the preliminary studies was to de-risk the insect repellent product for more cost-intensive Phase I SBIR studies. Insect repellent products must be registered with the EPA prior to sale; a process, which can be arduous and prohibitive due to the costs and timeline involved. The preliminary studies, therefore, involved considerable diligence to identify an EPA registered DEET formulation that would be compatible with the present dispenser/applicator technology and not require further regulatory approval. A 25% DEET formulation (EPA Registration Number 498-148) from the contract manufacturer Chase Products Inc. (IL) was selected due to aerosol compatibility and extended protection (5 hrs) offered by the formulation.

Consumer Product Research: Consumer feedback is an important metric for designing products that will be commercially viable, and well received. To assess the consumer perception and purchase intent of the insect repellent, a product viability test was undertaken by Hit Laboratories (Seattle, Wash.). In this study, 439 domestic participants from an online respondent database (5% confidence interval 95% level) were presented with information about the product including digital product images, a short instructional video, and a questionnaire. In this study, “51% of respondents indicated they would prefer to purchase the product or something similar from a local store—an unusually high number, suggesting suitability as a retail product.”Respondents with purchase intent ranked product features in order of importance to them, with the present dispenser/applicator being “the most important reason to purchase the product over similar products in the category”.

viii. Formula Attributes: The insect repellent discussed herein is an EPA registered (EPA Registration Number 498-148) ethanolic mixture containing 25% DEET (w/w), and provides continuous protection against mosquitoes for up to 5 hrs.³⁵ It can be sprayed on exposed skin or clothing to repel mosquitoes, deer ticks, blackflies, chiggers, deerflies, fleas, gnats, and stable flies. The formula is readily prepared at Chase Products Inc (Broadview, Ill.) using standard low-cost formulation procedures. The aerosolized fonnula, when delivered to the skin through the present dispenser/applicator device as a fine mist, dries within 1 minute. The formula has passed thermal bulk and in-package stability studies, and will have a Date of Minimum Durability of 36 months.

### Sample Formulation (% w/w)

35-40% Ethanol
20-25% DEET
15-20% n-Butane
5-10% Propane
1-5% n-Octylbicyclopheptene dicarboximide
1-5% low odor mineral spirits
1-5% di-N-propyl isocinchomeronate

### REFERENCES

1. Schon, M. P.; Boehncke, W. H., Psoriasis, The New England journal of medicine 2005, 352 (18), 1899-912.

2. Rachakonda, T. D.; Schupp, C. W.; Armstrong, A. W., Psoriasis prevalence among adults in the United States. J Am Acad Dermatol 2014, 70 (3), 512-6.

3. Kurd, S. Gelfand, M., The prevalence of previously diagnosed and undiagnosed psoriasis in US adults: results from NHANES 2003-2004. J Am Acad Dermatol 2009, 60 (2), 218-24.

4. Brezinski, E. A.; Dhillon, J. S.; Armstrong, A. W., Economic Burden of Psoriasis in the United States: A Systematic Review. JAMA dermatology 2015, 151 (6), 651-8.

5. Samarrasekera, E. J.; Sawyer, L.; Wonderling, D.; Tucker, R.; Smith, C. H., Topical therapies for the treatment of plaque psoriasis: systematic review and network, meta-analysis. Br J Dermatol 2013, 168 (5), 954-67.

6. Lebwohl, M. G.; Kavanaugh, A.; Armstrong W. A.; Van Voorhees, A. S., US Perspectives in the Management of Psoriasis and Psoriatic Arthritis: Patient and Physician Results from the Population-Based Multinational Assessment of Psoriasis and Psoriatic Arthritis (MAPP) Survey. American journal of clinical dermatology 2016, 17 (1), 87-97.

7. Beutner, K.; Chakrabarty, A.; Lemke, S.; Yu, K., An intra-individual randomized safety and efficacy comparison of clobetasol propionate 0.05% spray and its vehicle in the treatment of plaque psoriasis. Journal of drugs in dermatology: JDD 2006, 5 (4) 357-60.

8. Lasjls, Clobex Spray. National Psoriasis Foundation: inspire Talk Psoriasis, 2008; Vol. 2016, p Psoriasis and psoriatic arthritis support group and discussion community.

9. Northerngal, Clobex Spray. National Psoriasis Foundation: Inspire Talk Psoriasis, 2008; Vol. 2016, p Psoriasis and psoriatie arthritis support group and discussion community.

10. HouseFixer, Clobex Spray. National Psoriasis Foundation: Inspire Talk Psoriasis, 2016; Vol. 2016, p Psoriasis and psoriatic arthritis support group and discussion community.

11. luvs2golf2, Clobex Spray. National Psoriasis Foundation: Inspire Talk Psoriasis, 2013; Vol. 2016, p Psoriasis and psoriatic arthritis support group and discussion community.

12. Food Drug Administration Center for Drug Evaluation and Research (COER). Guidance for Industry: Applications Covered by Section 505(b)(2). 1999.

13. Food Drug Adminstration Center for Drug Evaluation and Research (COER), Center for Biologics Evaluation and Research (CBER). Guidance for Industry: Formal Meetings Between the FDA and Sponsors or Applicants. 2009, Revision l.

14. Parisi, R.; Symmons, D. P.; Griffiths, C. E.; Ashcroft, D. M., Global epidemiology of psoriasis: a systematic review of incidence and prevalence. The journal of investigative dermatology 2013, 133 (2), 377-85.

15. Gelfand J. M.; Feldman, S. R.; Stern, R. S.; Thomas, J.; Rolstad, T.; Margolis, D. J., Determination of quality of life in patients with psoriasis: a study from the US population. Journal of the American Academy of Dermatolagy 2004, 51 (5), 704-8.

16. Rosenberg, E. W.; Glenn, T. M.; Skinner, R. B.; Noah, P. W., Composition and method for treatment of psoriasis. Google Patents: 1999.

17. Seidel, W. E., Formulation containing a carrier, active ingredient, and surfactant for treating skin disorders. Google Patents: 1999.

18. Beutner, K.; Chakrabarty, A; Lemke, S.; Yu, K., An intra-individual randomized safety and efficacy comparison of clobetasol propionate 0.05% spray and its vehicle in the treatment of plaque psoriasis. Journal of drugs in dernmuology: JDD 2006, 5 (4), 357-360.

19. Lebwohl, M.; Sherer, D.; Washenik, K.; Krueger, G. G.; Menter, A; Koo, J.; Feldman, S. R., A randomized, double-blind, placebo-controlled study of clobetasol propionate 0.05% foam in the treatment of nonscaip psoriasis. Int J Dermatol 2002, 41 (5), 269-74.

20. Menter, A; Korman, N. J.; Elmets, C. A; Feldman, S. R.; Gelfand J. M.; Gordon,

K. B.; Gottlieb, A; Koo, J. Y.; Lebwohl, M.; Lim, H. W.; Van Voorhees, A. S.; Beutner, K. R.; Shushan, R.; American Academy, of D., Guidelines of care for the management of psoriasis and psoriatic arthritis. Section 3. Guidelines of care for the management and treatment of psoriasis with topical therapies. J Am Acad Dermatol 2009, 60 (4), 643-59.

21. Feldman, S. R.; Yentzer, B. A, Topical clobetasol propionate in the treatment of psoriasis: a review of newer formulations. American journal of clinical dermatology 2009, 10 (6), 397-406.

22. Housman, T. S.; Mellen, B. G.; Rapp, S. R.; Fleischer, A B., Jr.; Feldman, S. R., Patients with psoriasis prefer solution and foam vehicles: a quantitative assessment of vehicle preference. Cutis 2002, 70 (6), 327-32.

23. Menter, M. A; Caveney, S. W.; Gottschalk, R. W., Impact of clobetasol propionate 0.05% spray on health-related quality of life in patients with plaque psoriasis. J Drugs Dermatol 2012, 11 (11), 1348-54.

24. Puig, L.; Carrascosa, J. M.; Belinchon, l.; Fernandez-Redondo, V.; Carretero, G.; Ruiz-Carrascosa, J. C.; Careaga, J. M.; de la Cueva, P.; Garate, M. T.; Ribera M., Adherence and patient satisfaction with topical treatment in psoriasis, and the use, and organoleptic properties of such treatments: a Delphi study with an expert panel and members of the Psoriasis Group of the Spanish Academy of Dermatology and Venereology. Aeies dermo-sifi/iograficas 2013, 104 (6), 488-96.

25. Del Rosso, J. Q.; Bikowski, J. B.; Baum, E.; Smith, J.; Hawkes, S.; Benes, V.; Bhatia, N., A closer look at truncal acne vulgaris: prevalence, severity, and clinical significance. J Drugs Dermato/2007, 6 (6), 597-600.

26. Butler, D. C.; Koo, J. Y. M., Psoriasis Therapy in the Geriatric Population. In Adrvnces in Geriatric Dermatology, Chang, A. L. S., Ed. Springer International Publishing Switzerland: 2015.

27. American Mosquito Control Association Repellents. http://www.mosquito.org/repellents.

28, Petersen, L. R.; Jamieson, D. J.; Powers, A. M.; Henein, M. A, Zika Virus. New England Journal of Medicine 2016, 374 (16), 1552-1563.

29. Duffy, M. R.; Chen, T. H.; Hancock, W. T.; Powers, A M.; Kool, J. L.; Lanciotti, R. S.; Pretrick, M.; Marfel, M.; Holbauer, S.; Dubray, C.; Guillaumot, L.; Griggs, A; Bel, M.; Lambert, A J.; Laven, J.; Kosoy, 0.; Panella, A; Biggerstaff, B. J.; Fischer, M.; Hayes,

E. B., Zika virus outbreak on Yap Island, Federated States of Micronesia. N Eng J. Med 2009, 360 (24), 2536-43.

30. Mlakar, J.; Korva, M.; Tut N.; Popovic, M.; Poljsak-Prijatelj, M.; Mraz, J.; Kolenc, M.; Resman Rus, K.; Vesnaver Vipotnik, Fabjan Vodusek, V.; Vizjak, A; Pizem, J.; Petrovec, M.; Avsic Zupanc, T., Zika Virus Associated with Microcephaly. New England Journal of Medicine 2016, 374 (10), 951-958.

31. Driggers, R. W,; Ho, C. Y.; Korhonen, E. M.; Kuivanen, S.; Jaaskelainen, A J.; Smura, T.; Rosenberg, A; Hill, D. A; DeBiasi, R. L.; Vezina, G.; Timofeev, J.; Rodriguez, F. J.; Levanov, L.; Razak, J.; Iyengar, P.; Hennenfent, A; Kennedy, R.; Lanciotti, R.; du Plessis, A; Vapalahti, 0., Zika Virus Infection with Prolonged Maternal Viremia and Fetal Brain Abnormalities, N Eng/J Med 2016, 374 (22), 2142-51.

32. Rasmussen, S. A; Jamieson, D. J.; Henein, M. A; Petersen, L. R., Zika Virus and Birth Defects—Reviewing the Evidence for Causality. N Eng/J Med 2016, 374 (20), 1981-7.

33. Cao-Lormeau, V.-M.; Blake, A; Mons, S.; Lastere, S.; Roche, C.; Vanhomwegen, J.; Dub, T.; Baudouin, L.; Teissier, A; Larre, P.; Vial, A-L.; Decam, C.; Choumet, V.; Halstead, S. K.; Willison, H. J.; Musset, L.; Manuguerra, J.-C.; Despres, P.; Fournier, E.; Mallet, H.-P.; Musso, D.; Fontanet, A; Neil, J.; Ghawche, F.; Guillain-Barr&#xe9; Syndrome outbreak associated with Zika virus infection in French Polynesia: a case-control study. The Lancet 387 (10027), 1531-1539.

34. Centers for Disease Control and Prevention Prevent Mosquito Bites.

http://cdc.gov/zika/prevention/prevent-mosquito-bites.html.

35. Fradin, M. S.; Day, J. F., Comparitive efficacy of insect repellents against mosquito bites, New England Journal of Medicine 2002, 347 (1), 13-18.

36. DuPont, DuPont Announces 2016 Packaging Innovation Award Winners. http://www.dupont.com/industries/packaging-and-printing/media/press-releases/2016-dupont-packaging-awards-winners.html (accessed Sep. 2, 2016).

1. An apparatus for delivering liquid, fluid, solid, colloidal suspension, or gas, said apparatus comprising:
a supply canister configured to contain the liquid, fluid, solid, colloidal suspension, or gas;
a cap attached to an upper end of said supply canister;
a hinged or rotatable ball joint mechanism;
an extension applicator arm having a first end attached to an upper end of said supply canister and a free second end, said first end being attached to said upper end of said supply canister by said hinged or rotatable ball joint mechanism to allow said extension applicator arm to be selectively pivoted with respect to said supply canister, said extension applicator arm defining a chamber or conduit extending longitudinally through said extension applicator arm;
an actuator operatively mounted to said supply canister and configured to enable flow of the liquid fluid, solid, colloidal suspension, or gas from said supply canister; and
a tube having one end connected to said actuator, said tube extending from said actuator through said hinged or rotatable ball joint mechanism and into said chamber or conduit of said extension applicator arm.

2. The apparatus defined in claim 1 wherein said extension applicator arm is collapsible and is formed of telescoping sections or parts.

3. The apparatus defined in claim 1 wherein said extension applicator arm includes a flexible hose or tube.

4. The apparatus defined in claim 3 wherein said extension applicator arm includes a rigid handpiece extending from an end of the flexible hose or tube.

5. The apparatus defined in claim 3 wherein said flexible hose or tube is provided at a free end with a nozzle or dispensing aperture, further comprising a rod attached at a distal end to said free end of said flexible hose or tube for use as a handle to position said free end and concomitantly said nozzle or dispensing aperture.

6. The apparatus defined in claim 1 wherein said extension applicator arm is formed of a multiplicity of rigid segments or tubular elements pivotably connected to one another to enable reconfiguration of said extension applicator arm by the application of manual bending force.

7. The apparatus defined in claim 1, further comprising an applicator pad removably affixed to a distal or free end of said extension applicator arm.

8. The apparatus defined in claim 7 wherein said applicator pad is made of porous foam material.

9. The apparatus defined in claim 7 wherein said applicator pad has an array or holes or aperture communicating with said tube for dispensing the liquid fluid, solid, colloidal suspension, or gas from said supply canister.

10. The apparatus defined in claim 1 wherein said supply canister houses a dip tube extending to a bottom of said supply canister, said dip tube being attached to a 360° valve configured for enabling spraying from said extension applicator arm when said supply canister is inverted.

11. The apparatus defined in claim 1 wherein said tube has another end disposed at said second end of said extension applicator arm to carry the liquid fluid, solid, colloidal suspension, or gas from said supply canister to said second end of said extension applicator arm.

12. An apparatus for delivering liquid, fluid, solid, colloidal suspension, or gas, comprising:
a supply canister adapted to contain the liquid, fluid, solid, colloidal suspension, or gas;
a flexible applicator member connected at one end to said supply canister and provided at an opposite end with at least one outlet, said flexible applicator member being the only structure between said supply canister and said at least one outlet so that said flexible applicator member may take any of a multiplicity of different shapes or configurations between said supply canister and said at least one outlet; and
an actuator operatively connected to said supply canister and said flexible applicator member, said actuator adapted to enable flow of the liquid fluid, solid, colloidal suspension, or gas from said supply canister and into flexible applicator member.

13. The apparatus defined in claim 12 wherein said flexible applicator member is taken from the group consisting of a tube, hose and reconfigurable jointed tubular member.

14. The apparatus defined in claim 12 wherein said flexible applicator member is connected to said supply canister via a hinged or rotatable ball joint mechanism.

15. The apparatus defined in claim 1 wherein said actuator takes the form of a manual or mechanical pump spray actuator.

16. The apparatus defined in claim 15, further comprising a dip tube extending from said manual or mechanical pump spray actuator to a bottom of said supply canister, said hinged or rotatable ball joint mechanism being attached to said manual or mechanical pump spray actuator and including a 360° ball valve.

17. The apparatus defined in claim 12 wherein said at least one outlet is provided in a replaceable nozzle element attached to said flexible applicator member at a free end thereof.

18. The apparatus defined in claim 12, further comprising a cap attached to an upper end of said supply canister over said actuator.

19. An apparatus or device for delivering liquid, fluid, solid, colloidal suspension, or gas comprising:
a. a supply canister for housing fluid, solid, colloidal suspension, or solid;
b. a propellant mechanism disposed at least partially in said supply canister and attached thereto, said propellant mechanism being taken from the group consisting of aerosol, bag-on-valve aerosol, or pump spray with a pump or with a dip tube;
c. a canister cap attached to said supply canister;
d. an actuator taken from the group consisting of a dispense button, a trigger, or a manual pump;
e. a hinged or ball joint connector mounted to the cap;
f. an extension applicator arm attached to the cap via the hinged or ball joint connector, the extension applicator arm being of a form taken from the group consisting of fixed length, telescoping, and flexible.

20. The apparatus or device defined in claim 17, wherein said propellant mechanism includes a non-aerosol pump with a 360° valve housing a ball that allows the valve to spray at any angle, even when inverted, the valve being connected to a dip tube that extends to the bottom of the canister and carries the canister contents to the valve.

21. The apparatus or device defined in claim 17 wherein said supply canister has a lateral cylindrical surface and said arm is movable between a first and a second position by said hinged or rotatable ball joint connector, said applicator arm being disposed adjacent to said lateral cylindrical surface in said first position in parallel with said lateral cylindrical surface.

22. An apparatus for delivering liquid, fluid, solid, colloidal suspension, or gas comprising:
a supply canister;
a bag or pouch disposed in said supply canister and configured to contain the liquid, fluid, solid, colloidal suspension, or gas;
a canister cap attached to the supply canister;
an extension applicator arm or wand having a proximal end and a distal end, the proximal end being pivotably attached to the canister cap;
a mechanical pump or actuator mounted to the supply canister and including a valve in a bag-on-valve configuration configured to release the liquid, fluid, solid, colloidal suspension, or gas from the bag or pouch; and
a tube at least partially housed in the extension applicator arm or wand, to carry the liquid, fluid, solid, colloidal suspension, or gas to a distal end of the extension applicator arm or wand.
